(19) United States
(12) Patent Application Publication
(10) Pub. No.: US 2005/0220741 A1
(43) Pub. Date: Oct. 6, 2005

(54) Cosmetic composition comprising concave particles

(76) Inventor: Christophe Dumousseaux, Tokyo (JP)

Correspondence Address: Thomas L. Irving;FINNEGAN, HENDERSON, FARABOW,, GARRETT & DUNNER, L.L.P., 901 New York Avenue, N.W., Washington, DC, 20001-4413, US

(21) Appl. No.: 11/085,539

(22) Filed: Mar. 22, 2005

Related U.S. Application Data
(60) Provisional application No. 60/554,932, filed on Mar. 22, 2004.

(30) Foreign Application Priority Data
Mar. 22, 2004 (FR) 04 50563

(51) Int. Cl. A61K 7/025

(52) U.S. Cl. 424/64

(57) ABSTRACT

A cosmetic composition comprising a pulverulent phase is disclosed, wherein the pulverulent phase comprises concave particles and platelet-shaped particles. The present disclosure also relates to a process for making up and caring for keratinous substances, such as the skin, comprising the application of the composition to the keratinous substances.

This application claims benefit of U.S. Provisional Application No. 60/554,932, filed Mar. 22, 2004, and French Application No. 04/50563, filed Mar. 22, 2004, both of which are hereby incorporated by reference.

The present disclosure relates to a cosmetic composition, such as a cosmetic composition in the form of a compact powder, comprising concave particles and platelet-shaped particles. The present disclosure also relates to a process for making up or caring for human keratinous substances, such as skin, hair or nails, comprising the application of the composition to the keratinous substances.

The composition according to the present disclosure can be a composition for making up or caring for the skin and can be provided in the form of a blusher, an eyeshadow, a face powder, a foundation, a concealer, a product for making up the body, a product for caring for the face, a product for caring for the body or an antisun product. For example, the composition of the present disclosure can be a foundation composition.

Make-up powders generally comprise, on the one hand, a pulverulent phase comprising, in particular, pigments and fillers and, on the other hand, a fatty phase as a binder comprising fatty substances. The fatty phase is intended to confer a degree of density on the finished product, to bestow softness and an emollient property on the make-up product and to promote its adherence to the skin.

Some make-up compositions, such as foundations, eyeshadows and blushers, are provided in the form of a compact powder generally comprising a fatty phase, referred to as a binder, and a pulverulent phase comprising, for example, pigments and/or pearlescent agents and/or fillers.

The preparation of compact powders may raise numerous difficulties as the final composition should be sufficiently homogeneous and compact to prevent fragmentation brought about, for example, by impacts. The compact powder should ideally also be easy to remove in order to allow the user to apply the composition to keratinous substances, such as the skin.

Furthermore, pearlescent agents are commonly employed as coloring materials in make-up products. Pearlescent agents are generally provided in the form of platelets and, because of their shape and their size, may produce a dragging effect when the make-up is applied to the skin, thus rendering the application of the product unpleasant to the user and not promoting the satisfactory spreading of the product in a uniform way over the skin.

Thus, it would be desirable to be able to obtain a make-up composition which comprises platelet-shaped particles and which spreads easily, with good slip, over keratinous substances, such as the skin, and which, when it is provided in the form of a compact powder, can readily disintegrate with the finger or using a sponge.

The present inventor has discovered that such a composition can be obtained by combining platelet-shaped particles with particles of concave shape. The composition then may exhibit good slip, facilitating satisfactory distribution of the product over the surface of the skin and thus making it possible to obtain a make-up homogeneously distributed over the skin. Furthermore, when the composition is in the form of a compact powder, it may exhibit good disintegrating properties, allowing the user to readily take up, with the finger or with a sponge, the necessary amount of product to apply the make-up.

More specifically, the present disclosure relates to a cosmetic composition, such as a-composition in the form of a compact powder, comprising a pulverulent phase, wherein the pulverulent phase comprises concave particles and platelet-shaped particles.

The present disclosure also relates to a cosmetic process for making up or for the non-therapeutic treatment of keratinous substances, such as the skin, comprising the application to the keratinous substances, such as the skin, of a composition as defined above.

The composition according to the present disclosure comprises concave particles. These particles thus have a surface exhibiting a rounded interior. The concave particles may comprise particles of portions of hollow spheres composed of a material, such as an organosilicone material.

In at least one embodiment, the concave particles have a mean diameter ranging from 0.05 μm to 10 μm.

The portions of hollow spheres used in the composition according to an embodiment of the present disclosure can have the shape of truncated hollow spheres exhibiting a single orifice communicating with their central cavity and having a transverse cross section with the shape of a horseshoe or arch.

The organosilicone material can be a crosslinked polysiloxane with a three-dimensional structure. In one embodiment, the polysiloxane with a three-dimensional structure may be composed of units of formula (I): SiO₂, and of formula (II): R¹SiO_{1.5}, wherein R¹ denotes an organic group having a carbon atom directly connected to the silicon atom. The organic group can be a reactive organic group or an unreactive organic group. In one embodiment, the organic group is an unreactive organic group.

The unreactive organic group can be a C₁-C₄ alkyl group, such as a methyl, ethyl, propyl or butyl group, or a phenyl group. In one embodiment, the unreactive organic group is a methyl group.

The reactive organic group can be an epoxy group, a (meth)acryloyloxy group, an alkenyl group, a mercaptoalkyl, aminoalkyl or haloalkyl group, a glyceroxy group, a ureido group or a cyano group. In one embodiment, the reactive organic group can be an epoxy group, a (meth)acryloyloxy group, an alkenyl group or a mercaptoalkyl or aminoalkyl group. The reactive organic group may comprise from 2 to 6 carbon atoms, such as from 2 to 4 carbon atoms.

Among the epoxy groups that may be used, non-limiting mention may be made of a 2-glycidoxyethyl group, a 3-glycidoxypropyl group or a 2-(3,4-epoxycyclohexyl)propyl group.

Among the (meth)acryloyloxy groups that may be used, non-limiting mention may be made of a 3-methacryloyloxypropyl group or a 3-acryloyloxypropyl group.

Among the alkenyl groups that may be used, non-limiting mention may be made of a vinyl, allyl or isopropenyl group.

Among the mercaptoalkyl groups that may be used, non-limiting mention may be made of a mercaptopropyl or mercaptoethyl group.

Among the aminoalkyl groups that may be used, non-limiting mention may be made of a 3-[(2-aminoethyl)amino]propyl group, a 3-aminopropyl group or an N,N-dimethylaminopropyl group.

Among the haloalkyl groups that may be used, non-limiting mention may be made of a 3-chloropropyl group or a trifluoropropyl group.

Among the glyceroxy groups that may be used, non-limiting mention may be made of a 3-glyceroxypropyl group or a 2-glyceroxyethyl group.

Among the ureido groups that may be used, non-limiting mention may be made of a 2-ureidoethyl group.

Among the cyano groups that may be used, non-limiting mention may be made of a cyanopropyl or cyanoethyl group.

In one embodiment of the present disclosure, in the unit of formula (II), R¹ denotes a methyl group.

In a further embodiment of the present disclosure, the organosilicone material comprises the units (I) and (II) according to a unit (I)/unit (II) molar ratio ranging from 30/70 to 50/50, such as from 35/65 to 45/55.

The organosilicone particles can be capable of being obtained according to a process comprising:

(a) introducing, into an aqueous medium, in the presence of at least one hydrolysis catalyst and optionally of at least one surfactant, a compound (III) of formula SiX4 and a compound (IV) of formula RSiY₃, wherein
X and Y denote, independently of one another, a C₁-C₄ alkoxy group, an alkoxyethoxy group including a C₁-C₄ alkoxy group, a C₂-C₄ acyloxy group, an N,N-dialkylamino group including a C₁-C₄ alkyl group, a hydroxyl group, a halogen atom or a hydrogen atom, and
R denotes an organic group comprising a carbon atom connected directly to the silicon atom; and

(b) bringing the mixture resulting from stage (a) into contact with an aqueous solution including at least one polymerization catalyst and optionally at least one surfactant, at a temperature of between 30 and 85° C., for at least two hours.

Stage (a) corresponds to a hydrolysis reaction and stage (b) corresponds to a condensation reaction.

In stage (a), the molar ratio of the compound (III) to the compound (IV) generally ranges from 30/70 to 50/50, such as from 35/65 to 45/45. In one embodiment, the molar ratio of the compound (III) to the compound (IV) is 40/60. The ratio by weight of the water to the total of the compounds (III) and (IV) may range from 10/90 to 70/30. The order of introduction of the compounds (III) and (IV) generally depends on their rate of hydrolysis. The temperature of the hydrolysis reaction generally ranges from 0 to 40° C. and usually does not exceed 30° C. in order to prevent premature condensation of the compounds.

For the X and Y groups of the compounds (III) and (IV), non-limiting mention may be made of the following groups:
C₁-C₄ alkoxy groups such as the methoxy or ethoxy groups;
alkoxyethoxy groups including a C₁-C₄ alkoxy group, such as the methoxyethoxy or butoxyethoxy groups;
C₂-C₄ acyloxy groups such as the acetoxy or propionyloxy groups;
N,N-dialkylamino group including a C₁-C₄ alkyl groups, such as the dimethylamino or diethylamino groups; and
halogen atoms such as the chlorine or bromine atoms.

Among the compounds of formula (III) that may be used, non-limiting mention may be made of tetramethoxysilane, tetraethoxysilane, tetrabutoxysilane, trimethoxyethoxysilane, tributoxyethoxysilane, tetraacetoxysilane, tetrapropioxysilane, tetra(dimethylamino)silane, tetra(diethylamino)silane, silanetetraol, chlorosilanetriol, dichlorodisilanol, tetrachlorosilane or chlorotrihydrosilane. In one embodiment, the compound of formula (III) is chosen from tetramethoxysilane, tetraethoxysilane, and tetrabutoxysilane, and the mixtures thereof.

The compound of formula (III) results, after the polymerization reaction, in the formation of the units of formula (I).

The compound of formula (IV) results, after the polymerization reaction, in the formation of the units of formula (II).

The R group in the compound of formula (IV) has the meaning as described for the R¹ group for the compound of formula (II).

Among the compounds of formula (IV) comprising an unreactive organic group R that may be used in the present disclosure, non-limiting mention may be made of methyltrimethoxysilane, ethyltriethoxysilane, propyltributoxysilane, butyltributoxysilane, phenyltrimethoxyethoxysilane, methyltributoxyethoxysilane, methyltriacetoxysilane, methyltripropioxysilane, methyltri(dimethylamino)silane, methyltri(diethylamino)silane, methylsilanetriol, methylchlorodisilanol, methyltrichlorosilane or methyltrihydrosilane.

Among examples compounds of formula (IV) comprising a reactive organic group R that may be used in the present disclosure, non-limiting mention may be made of:
silanes having an epoxy group, such as (3-glycidoxypropyl)trimethoxysilane, (3-glycidoxypropyl)triethoxysilane, [2-(3,4-epoxycyclohexyl)ethyl]trimethoxysilane, (3-glycidoxypropyl)methyldimethoxysilane, (2-glycidoxyethyl)methyldimethoxysilane, (3-glycidoxypropyl)dimethylmethoxysilane or (2-glycidoxyethyl)dimethylmethoxysilane;
silanes having a (meth)acryloyloxy group, such as (3-methacryloyloxypropyl)trimethoxysilane or (3-acryloyloxypropyl)trimethoxysilane;
silanes having an alkenyl group, such as vinyltrimethoxysilane, allyltrimethoxysilane or isopropenyltrimethoxysilane;
silanes having a mercapto group, such as mercaptopropyltrimethoxysilane or mercaptoethyltrimethoxysilane;
silanes having an aminoalkyl group, such as (3-aminopropyl)trimethoxysilane, (3-[(2-aminoethyl)amino]propyl)trimethoxysilane, (N,N-dimethylaminopropyl)trimethoxysilane or (N,N-dimethylaminoethyl)trimethoxysilane;
silanes having a haloalkyl group, such as (3-chloropropyl)trimethoxysilane or trifluoropropyltrimethoxysilane;
silanes having a glyceroxy group, such as (3-glyceroxypropyl)trimethoxysilane or di(3-glyceroxypropyl)dimethoxysilane;
silanes having a ureido group, such as (3-ureidopropyl)trimethoxysilane, (3-ureidopropyl)methyidimethoxysilane or (3-ureidopropyl)dimethylmethoxysilane; and
silanes having a cyano group, such as cyanopropyltrimethoxysilane, cyanopropylmethyldimethoxysilane or cyanopropyidimethylmethoxysilane.

In one embodiment of the present disclosure, the compound of formula (IV) comprising a reactive organic group R is chosen from silanes having an epoxy group, silanes having a (meth)acryloyloxy group, silanes having an alkenyl group, silanes having a mercapto group, and silanes having an aminoalkyl group.

Examples of compounds (III) and (IV) which are used in another embodiment of the present disclosure are tetraethoxysilane and methyltrimethoxysilane, respectively.

Use may independently be made, as hydrolysis and polymerization catalysts, of basic catalysts, such as sodium hydroxide, potassium hydroxide, sodium carbonate, sodium hydrogencarbonate or amines (such as ammonia, trimethylamine, triethylamine or tetraethylammonium hydroxide), or acidic catalysts chosen from organic acids, such as citric acid, acetic acid, methanesulphonic acid, p-toluenesulphonic acid, dodecylbenzenesulphonic acid or dodecylsulphonic acid, and inorganic acids, such as hydrochloric acid, sulphuric acid or phosphoric acid.

When it is present, the at least one surfactant used in the process described above may comprise a nonionic or anionic surfactant or a mixture of the two. Sodium dodecylbenzenesulphonate can be used as anionic surfactant. The end of the hydrolysis is marked by the disappearance of the products (III) and (IV), which are insoluble in water, and the production of a homogeneous liquid layer.

The condensation stage (b) can use the same catalyst as the hydrolysis stage or another catalyst chosen from those mentioned above.

On conclusion of this process, a suspension in water of fine organosilicone particles is obtained, which particles can optionally be separated subsequently from their medium. The process described above can thus comprise an additional stage of filtration, for example on a membrane filter, of the product resulting from stage (b), optionally followed by a stage of centrifuging the filtrate, intended to separate the particles from the liquid medium, and then by a stage of drying the particles. Other separation methods can, of course, be employed.

In one embodiment of the present invention, the particles obtained (or the spheres) have a mean diameter ranging from 0.05 to 10 μm.

The shape of the portions of hollow spheres obtained according to the above process and their dimensions will depend in particular on the method used to bring the products into contact in stage (b).

A somewhat basic pH and introduction under cold conditions of the polymerization catalyst into the mixture resulting from stage (a) will result in portions of hollow spheres with the shape of round-bottomed “bowls”, whereas a somewhat acidic pH and dropwise introduction of the mixture resulting from stage (a) into the hot polymerization catalyst will result in portions of hollow spheres having a transverse cross section with the shape of a horseshoe.

According to a one embodiment of the present disclosure, portions of hollow spheres with the shape of bowls are used. These can be obtained as disclosed in Japanese Patent Application No. JP-2003-128788.

Portions of hollow spheres with the shape of a horseshoe are also disclosed in Japanese Patent Application No. JP-A-2000-191789.

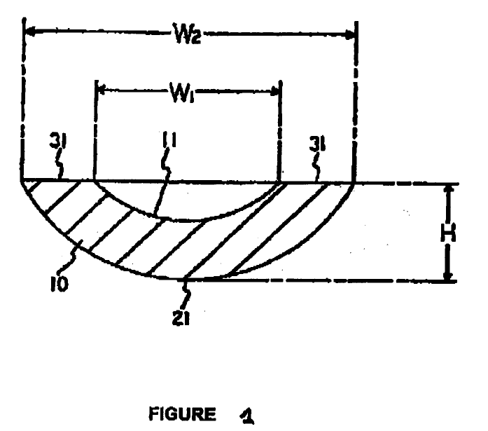

A concave particle formed of portions of spheres with the shape of a bowl is illustrated in transverse cross section in the appended FIG. 1.

As seen in FIG. 1, these concave portions are formed (in longitudinal cross section) of a small internal arc (11), of a large external arc (21) and of segments (31) which connect the ends of the respective arcs. The width (W1) between the two ends of the small internal arc (11) may range from 0.01 to 8 μm, such as from 0.02 to 6 μm, on average. The width (W2) between the two ends of the large external arc (21) may range from 0.05 to 10 μm, such as from 0.06 to 8 μm, on average. The height (H) of the large external arc (21) may range from 0.015 to 8 μm, such as from 0.03 to 6 μm, on average.

The dimensions mentioned above are obtained by calculating the mean of the dimensions of one hundred particles chosen on an image obtained with a scanning electron microscope.

Among the concave particles of portions of spheres which can be used according to the present disclosure, non-limiting mention may be made of:
particles composed of the crosslinked organosilicone TAK-110 (crosslinked methylsilanol/silicate polymer) from Takemoto Oil & Fat, with the shape of a bowl, with a width of 2.5 μm, a height of 1.2 μm and a thickness of 150 nm (particles sold under the name NLK-506 by Takemoto Oil & Fat);
particles composed of the crosslinked organosilicone TAK-110 (crosslinked methylsilanovsilicate polymer) from Takemoto Oil & Fat, with the shape of a bowl, with a width of 2.5 μm, a height of 1.5 μm and a thickness of 350 nm;
particles composed of the crosslinked organosilicone TAK-110 (crosslinked methylsilanovsilicate polymer) from Takemoto Oil & Fat, with the shape of a bowl, with a width of 0.7 μm, a height of 0.35 μm and a thickness of 100 nm; and
particles composed of the crosslinked organosilicone TAK-110 (crosslinked methylsilanovsilicate polymer) from Takemoto Oil & Fat, with the shape of a bowl, with a width of 7.5 μm, a height of 3.5 μm and a thickness of 200 nm.

The concave particles, such as the particles of portions of hollow spheres, can be present in the composition according to the present disclosure, such as the compact powder, in an amount ranging from 0.01% to 50% by weight, relative to the total weight of the composition, such as from 0.1% to 30% by weight with respect to the total weight of the composition. In one embodiment, the concave particles can be present in the composition in an amount ranging from 1% to 15% by weight relative to the total weight of the composition.

The term “platelet” is understood to mean, according to the present disclosure, particles with a shape characterized by three dimensions, a length, a width and a height, also referred to as thickness, the ratio of the greatest dimension to the thickness of which is greater than or equal to 5.

Within the meaning of the present disclosure, the term “greatest dimension of the platelets” is understood to mean the diameter of the sphere within which the said platelet is framed.

The platelet-shaped particles can be of parallelepipedal (rectangular or square surface), discoidal (circular surface) or ellipsoidal (oval surface) shape.

The platelet-shaped particles can be chosen from platelets of inorganic materials. In one embodiment, the platelets can be chosen from platelets of mica, sericite, glass (such as calcium borosilicate), silica, aluminum oxide and barium sulphate.

The platelet-shaped particles can be surface treated, such as covered with a layer of metal or of metal oxide.

Among the metals that may be used according to the present disclosure, non-limiting mention may be made of silver, aluminium, chromium, nickel, molybdenum, gold, copper, tin, magnesium and their mixtures (alloys). In one-embodiment, the metal may be silver, chromium, nickel, molybdenum and mixtures thereof.

Among the metal oxides that may be used according to the present disclosure, non-limiting mention may be made of titanium dioxide, iron oxides, zinc oxides or chromium oxide. In one embodiment, the metal oxide may be titanium dioxide.

In a further embodiment of the present disclosure, use may be made, as platelet-shaped particles, of:
the mica platelets sold under the names “Mica Concord 1000” by Sciama, “Cardre Mica 8” by Cardre, “Mica 40” by Eckart and “PDM-20 L” and “PDM-40 L” by Topy;
the sericite platelets sold under the names “Sericite FS” and “Sericite FSE” by Sanshin Mining, “NAI-S-100” and “Sericite S-152” by Miyoshi and “Synthetice FNK-100” by Topy;
the aluminium oxide platelets sold under the name “Luxelen FAO” by Asahi;

the silica platelets sold under the names “TSG 30A Flake”, “PTSG 30 A Flake” and “Silica Flake SG” by Nippon Sheet Glass;
the barium sulphate platelets sold under the name “Flake shaped Barium sulphate” by Sakai Chemical;
the mica platelets covered with titanium dioxide sold under the names “Flamenco”, “Duochrome” and “Cloisonne” by Engelhard and “Timiron” by Merck;
the synthetic mica platelets covered with titanium dioxide sold under the names “Prominence” by Nihon Koken and “Sunshine” by Sun Chemical;
the aluminium oxide platelets covered with titanium dioxide sold under the names “Xirona silver” and “Xirallic” by Merck;
the silica platelets covered with titanium dioxide sold under the names “Colorstream” and “Xirona” by Merck;
the glass platelets covered with a metallic layer; use may be made, for example, of the particles covered with silver sold under the names Microglass Metashine REFSX 2025 PS and GF 2140 by Toyal and the particles covered with nickel/chromium/molybdenum alloy sold under the names Crystal star GF 550 and GF 2525 by Toyal;
the glass platelets covered with titanium dioxide sold under the name “Reflecks” by Engelhard; and
the talc platelets covered with titanium dioxide sold under the name “Silseem” by Nihon Koken.

The platelet-shaped particles can also be surface treated with a hydrophobic treatment agent.

The hydrophobic treatment agent can be chosen from silicones, such as methicones, dimethicones or perfluoroalkylsilanes, fatty acids, such as stearic acid, metal soaps, such as aluminium dimyristate or the aluminium salt of hydrogenated tallow glutamate, perfluoroalkyl phosphates, perfluoroalkylsilanes, perfluoroalkylsilazanes, poly(hexafluoropropylene oxide)s, polyorganosiloxanes comprising perfluoroalkyl-perfluoropolyether groups, amino acids, N-acylated amino acids or their salts, lecithin, isopropyl triisostearyl titanate and the mixtures thereof.

The N-acylated amino acids can comprise an acyl group having from 8 to 22 carbon atoms, such as, for example, a 2-ethylhexanoyl, caproyl, lauroyl, myristoyl, palmitoyl, stearoyl or cocoyl group. The salts of these compounds can be chosen from the aluminium, magnesium, calcium, zirconium, zinc, sodium and potassium salts. The amino acid can be chosen from, for example, lysine, glutamic acid and alanine.

The term “alkyl” mentioned in the above-listed compounds, as defined herein, refers to an alkyl group having from 1 to 30 carbon atoms, such as an alkyl group having from 5 to 16 carbon atoms.

Hydrophobically-treated pigments are disclosed in European Application No. EP-A-1 086 683.

The platelet-shaped particles can also be flat fibers.

The term “fiber” should be understood as meaning an object with a length L and a diameter D such that L is much greater than D, D being the diameter of the circle within which the cross section (transverse cross section) of the fiber is framed. In one embodiment of the present disclosure, the ratio UD (or aspect ratio) ranges from 5 to 2500. In another embodiment of the present disclosure, the ratio UD ranges from 5 to 500. In a further embodiment, the ratio UD ranges from 5 to 150.

The-term “flat fiber” is understood to mean a fiber with a transverse cross section (cross section perpendicular to the axis of the direction of the length of the fiber) which exhibits a greatest length L1 and a smallest length L2 (L2 corresponds to the thickness of the fiber) such that L1/L2 (the ratio L1/L2 is also known as the flattening factor) is greater than or equal to 4, such as greater than 7. In one embodiment of the present disclosure, L1/L2 ranges from 4 to 15. In another embodiment of the present disclosure, L1/L2 ranges from 6 to 12. In a further embodiment of the present disclosure, L1/L2 ranges from 7 to 10. Thus, the transverse cross section of the fiber exhibits a flat shape. In one embodiment of the present disclosure, the greatest length L1 and the smallest length L2 respectively define axes X1 and X2 such that the axis X1 is substantially perpendicular to the axis X2. The greatest length L1 corresponds to the diameter D of the fiber, as mentioned above. Thus, the flat fibers can be provided in the ribbon or tagliatelle form.

The flat fibers can exhibit a transverse cross section of substantially rectangular, ovoid or ellipsoidal shape.

The fibers which can be used in the composition of the present disclosure can be fibers of synthetic or organic origin, such as fibers of synthetic polymer. They can be short or long, individual (or monofilament) or organized, for example plaited (or multifilament), and hollow or solid. In one embodiment, the fibers are solid. When the fibers are multifilament fibers, each filament can have a different chemical composition and can exhibit a different color: multifilament fibers exhibiting different colors are thus obtained. In one embodiment, the ends of the fibers are blunted and/or polished to prevent injury. In one embodiment of the present disclosure, the flat fibers are insoluble in water.

The flat fiber-can be twisted along the axis of the length L of the fiber. When the flat fiber is not twisted, it exhibits a color within a certain angle of view; outside this angle, the fiber is transparent or white in color. The twisted flat fiber, for its part, exhibits a color whatever the angle of observation.

In one embodiment of the present disclosure, the fibers have a length ranging from 1 μm to 10 mm, such as from 0.1 mm to 5 mm. In a further embodiment of the present disclosure, the fibers have a length ranging from 0.3 mm to 3.5 mm. Their transverse cross section (flat cross section) can be included within a circle with a diameter ranging from 2 nm to 500 μm, such as ranging from 100 nm to 100 μm. In a further embodiment, the transverse cross section can be within a circle with a diameter ranging from 1 μm to 70 μm. The weight or count of the fibers is often given in denier or decitex and represents the weight in grams per 9 km of yarn. In one embodiment, the fibers according to the present disclosure have a count ranging from 0.15 to 30 deniers. In a further embodiment, the fibers have a count ranging from 0.18 to 18 deniers.

The fibers can be fibers formed of rayon, polyamide (Nylon®), viscose, acetate, such as rayon acetate, poly(p-phenylene terephthalamide) or aramide, such as Kevlar®, acrylic polymer, such as poly(methyl methacrylate) or poly(2-hydroxyethyl methacrylate), polyolefin, such as polyethylene or polypropylene, polytetrafluoroethylene (such as Teflon®), poly(vinyl chloride) or poly(vinylidene chloride), poly(vinylidene fluoride), poly(vinyl alcohol), polyacrylonitrile, polyurethane, polyesters, such as poly(ethylene terephthalate)s or poly(ethylene naphthalate)s, or polycarbonate.

Flat fibers are disclosed in International Patent Application No. WO-A-02/41851, the content of which is hereby incorporated by way of reference.

In one embodiment of the present disclosure, use may be made of flat fibers with a multilayer structure comprising alternating layers of polymers chosen from polyesters, acrylic polymers and polyamides, such as those disclosed in the European Patent Nos. EP-A-921217 and EP-A-686 858, and U.S. Pat. No. 5,472,798. Such fibers are sold under the names “Morphotex” and “Teijin Tetron Morphotex” by Teijin.

The platelet-shaped particles can be present in the composition according to the present disclosure in an amount ranging from 1% to 99% by weight, relative to the total weight of the composition. In one embodiment, the platelet-shaped particles can be present in an amount ranging from 5% to 90% by weight relative to the total weight of the composition, such as ranging from 10% to 80% by weight relative to the total weight of the composition.

In one embodiment of the present disclosure, the platelet-shaped particles and the concave particles are present in the composition according to the present disclosure in an amount such that the ratio by weight of platelet-shaped particles to concave particles ranges from 1 to 100, such as from 2 to 75. In a further embodiment of the present disclosure, the ratio by weight of platelet-shaped particles to concave particles ranges from 5 to 50.

In an embodiment of the present disclosure, the concave particles, such as the particles of portions of hollow spheres (including those made of organosilicone material), are present in the composition according to the present disclosure in an amount ranging from 1% to 50% by weight, relative to the total weight of the concave particles and the platelet-shaped particles. In another embodiment, the concave particles are present in an amount ranging from 2% to 45% by weight relative to the total weight of the concave particles and the platelet-shaped particles. In a-further embodiment, the concave particles are present in-an amount ranging from 5% to 40% by weight relative to the total weight of the concave particles and the platelet-shaped particles.

The composition according to the present disclosure can comprise an additional coloring material different from the platelet-shaped particles described above and which can be chosen from pigments and glitter.

The term “pigments” should be understood as meaning white or colored, inorganic or organic particles of any shape which are insoluble in the physiological medium and which are intended to color the composition.

The pigments can be white or colored and inorganic and/or organic. Among organic pigments that may be used according to the present disclosure, non-limiting mention may be made of titanium dioxide, optionally surface treated, zirconium or cerium oxides, as well as zinc, (black, yellow or red) iron or chromium oxides, manganese violet, ultramarine blue, chromium hydrate and ferric blue, or metal powders, such as aluminium powder or copper powder.

Among organic pigments that may be used according to the present disclosure, further non-limiting mention may be made of carbon black, pigments of D & C type and lakes based on cochineal carmine and on barium, strontium, calcium or aluminium.

The pigments can be present in the composition in an amount ranging from 0.1% to 15% by weight, relative to the total weight of the composition. In one embodiment, the pigments can be present in an amount ranging from 0.5% to 12% by weight relative to the total weight of the composition. In a further embodiment, the pigments can be present in an amount ranging from 1% to 10% by weight relative to the total weight of the composition.

The composition according to the present disclosure, such as a compact powder, can also comprise a liquid fatty phase (liquid at ambient temperature (25° C.)) generally referred to as binder. This liquid fatty phase can comprise an oil generally used in compact powders.

The oil can be chosen from oils conventionally used as a binder in compact powders. Among the additional oils which can be used, non-limiting mention may be made of mink oil, turtle oil, soybean oil, grape seed oil, sesame oil, maize oil, rapeseed oil, sunflower oil, cottonseed oil, avocado oil, olive oil, castor oil, jojoba oil or groundnut oil; hydrocarbon oils, such as liquid paraffins, squalane, liquid petrolatum or polydecene; fatty esters, such as isopropyl myristate, isopropyl palmitate, butyl stearate, isodecyl stearate, hexyl laurate, isononyl isononanoate, 2-ethylhexyl palmitate, 2-hexyldecyl laurate, 2-octyldecyl palmitate, 2-octyldodecyl myristate, 2-octyldodecyl lactate, di(2-ethylhexyl) succinate, diisostearyl malate, glyceryl triisostearate or diglyceryl triisostearate; silicone oils, such as polymethylsiloxanes, polymethylphenylsiloxanes, polysiloxanes modified by fatty acids, fatty alcohols or polyoxyalkylenes, fluorinated silicones or perfluorinated oils; oleic acid, linoleic acid, linolenic acid or isostearic acid; or higher fatty alcohols, such as cetanol or oleyl alcohol.

The liquid fatty phase can be present in an amount ranging from 0.1% to 13% by weight, relative to the total weight of the composition. In one embodiment, the liquid fatty phase can be present in an amount ranging from 0.1% to 10% by weight relative to the total weight of the composition. In a further embodiment of the present disclosure, the liquid fatty phase can be present in an amount ranging from 0.1% to 8% by weight relative to the total weight of the composition.

The composition according to the present disclosure, such as a compact powder, can also comprise additional fillers different from the platelet-shaped particles described above. The term “fillers” should be understood as meaning colorless or white, inorganic or synthetic particles which are insoluble in the medium of the composition, whatever the temperature at which the composition is manufactured.

The additional fillers can be inorganic or organic, and can be of spherical or oblong shape, whatever the crystallographic form (for example sheet, cubic, hexagonal, orthorhombic, and the like). Non-limiting mention may be made of talc, mica, silica, kaolin, powders formed of polyarnide (Nylon®), of poly-β-alanine and of polyethylene, powders formed of polyurethane, powders formed of tetrafluoroethylene polymers (Teflon®), lauryllysine, starch, boron nitride, polymeric hollow microspheres, such as those of poly(vinylidene chloride)/acrylonitrile, for example Expancel® (Nobel Industrie), or of acrylic acid copolymers, silicone resin microbeads (Tospearls® from Toshiba, for example), particles formed of polyorganosiloxane elastomers, precipitated calcium carbonate, magnesium carbonate, basic magnesium carbonate, hydroxyapatite, hollow silica microspheres, glass or ceramic microcapsules, or metal soaps derived from organic carboxylic acids having from 8 to 22 carbon atoms, such as from 12 to 18 carbon atoms, for example zinc stearate, magnesium stearate, lithium stearate, zinc laurate or magnesium myristate.

The additional fillers can be present in the composition in an amount ranging from 0.1% to 90% by weight, relative to the total weight of the composition. In one embodiment, the additional fillers can be present in an amount ranging from 1% to 80% by weight relative to the total weight of the composition. In a further embodiment of the present disclosure, the additional fillers can be present in an amount ranging from 5% to 70% by weight relative to the total weight of the composition.

The composition can comprise other conventional cosmetic ingredients which can be chosen, for example, from antioxidants, fragrances, preservatives, neutralizing agents, surfactants, waxes, sunscreens, vitamins, moisturizing agents, self-tanning compounds or antiwrinkle active principles.

Of course, a person skilled in the art will take care to choose this or these optional additional compounds and/or their amounts such that the beneficial properties of the composition according to the present disclosure are not, or not substantially, detrimentally affected by the envisaged addition.

The composition according to the present disclosure can be an anhydrous composition, that is to say a composition comprising less than 2% by weight of water, such as less than 0.5% of water or devoid of water, the water not being added during the preparation of the composition but corresponding to the residual water introduced by the ingredients mixed.

The composition can be prepared by mixing the ingredients of the pulverulent phase (organosilicone particles, fillers and pigments) and by then adding the fatty phase with stirring, the mixture subsequently being milled, sieved, then poured into a dish and compacted.

The milled and sieved mixture of the pulverulent phase and of the fatty phase is compacted using a press, such as by applying a pressure ranging from 0.5 MPa to 10 MPa. In one embodiment of the present disclosure, the milled and sieved mixture of the pulverulent phase and of the fatty phase is compacted by applying a pressure ranging from 1 MPa to 5 MPa.

The composition thus obtained is provided in the form of a compact powder.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific example are reported as precisely as possible. Any numerical value, however, inherently contains certain errors necessarily resulting from the standard deviation found in their respective testing measurements.

The present disclosure is illustrated in more detail by the non-limiting examples described below.

### EXAMPLE 1

A compact powder having the following composition was prepared:

The contents are expressed as % by weight.

| | |
|---|---|
| Talc | 39.9 |
| Sericite platelets(3) | 33.25 |
| Bowl-shaped hemispherical particles(1) with a mean | 5 |
| width of 2.5 μm, with a thickness of 0.15 μm and with a | |
| height of 1.2 μm, composed of the organosilicone *TAK- | |
| 110 from Takemoto Oil & Fat | |
| Titanium dioxide | 5.2 |
| Mica platelets(2) | 4.75 |
| Iron oxide | 2.1 |
| Zinc stearate | 0.95 |
| Liquid paraffin | 3.8 |
| Phenyl trimethicone | 4.75 |
| Preservatives | 0.3 |

*crosslinked methylsilanol/silicate polymer
(1) particles sold under the name NLK-506 by Takemoto Oil & Fat
(2) sold under the name “Mica Concord 1000” by Sciama
(3) sold under the name “NAI-S-100” by Miyoshi

The composition was prepared by mixing all the powders and by then adding the binder (oils) thereto. This mixture was subsequently milled and sieved until a homogeneous mixture was obtained. 14 g of this mixture were placed in a dish and then pressed under a pressure of 2 MPa.

A compact powder which easily disintegrates using a sponge was thus obtained. The powder withdrawn slipped and spread well over the skin, with softness, and made it possible to obtain a make-up with good coverage.

### EXAMPLE 2

Six mixtures of bowl-shaped hemispherical particles, identical to those used in the preceding Example 1, and of mica platelets, sold under the name “Mica Concord 1000” by Sciama, were prepared in varied proportions by weight. For each mixture, the coefficient of friction was measured using a tribometer (device for measuring coefficient of friction) sold under the trade name “KES-E Friction Tester” by Katotech, equipped with a probe having a weight of 25 g, the rate of displacement of the probe being adjusted to a rate of 1 mm/s. For each mixture, a layer of the mixture with a thickness of approximately 1 mm was spread over a glass slide covered with a double-sided cellotape sold under the name “Nistack NW-20” by Nichiban. The coefficient of friction of each mixture was then measured with the device: the probe of the device moved while rubbing in the layer of the mixture deposited on the glass slide. The mean value of the dynamic coefficient of friction was calculated as the mean of the values measured between the second and the fifth successive pass of the probe over the same sample.

The following results were obtained:

| | | Organosilicone | |
|---|---|---|---|
| | Mica platelets | particles | Mean value of the |
| Examples | (% by weight) | (% by weight) | coefficient of friction |
| A | 100 | 0 | 0.77 |
| B | 95 | 5 | 0.66 |
| C | 90 | 10 | 0.63 |
| D | 80 | 20 | 0.59 |
| E | 60 | 40 | 0.56 |
| F | 0 | 100 | 0.68 |

It was found that the mixtures B, C, D and E, comprising from 5% to 40% of organosilicone particles, exhibited a lower coefficient of friction than those of the mixtures A and F. Thus, the mixture of mica platelets and of organosilicone particles exhibited better slip properties than those of each of the particles used alone.

1. A cosmetic composition comprising a pulverulent phase, wherein the pulverulent phase comprises concave particles and platelet-shaped particles.

2. The composition according to claim 1, wherein the concave particles are in the form of portions of hollow spheres.

3. The composition according to claim 1, wherein the concave particles have a mean diameter ranging from 0.05 μm to 10 μm.

4. The composition according to claim 2, wherein the particles of portions of hollow spheres have a transverse cross section with the shape of a horseshoe or arch.

5. The composition according to claim 2, wherein the particles of portions of hollow spheres are composed of an organosilicone material.

6. The composition according to claim 5, wherein the organosilicone material is a crosslinked polysiloxane with a three-dimensional structure comprising units of formula (I): SiO₂, and of formula (II): R¹SiO_{1.5}, wherein R¹ is an organic group having a carbon atom directly connected to the silicon atom.

7. The composition according to claim 6, wherein the organic group is a reactive organic group or an unreactive organic group.

8. The composition according to claim 6, wherein the organic group is an unreactive organic group

9. The composition according to claim 8, wherein the unreactive organic group is chosen from a C₁-C₄ alkyl group and a phenyl group.

10. The composition according to claim 8, wherein the unreactive organic group is a methyl group.

11. The composition according to claim 7, wherein the reactive organic group is chosen from an epoxy group, a (meth)acryloyloxy group, an alkenyl group, a mercaptoalkyl, aminoalkyl or haloalkyl group, a glyceroxy group, a ureido group and a cyano group.

12. The composition according to claim 11, wherein the reactive organic group is chosen from an epoxy group, a (meth)acryloyloxy group, an alkenyl group, a mercaptoalkyl group and aminoalkyl group.

13. The composition according to claim 6, wherein R¹ is a methyl group.

14. The composition according to claim 6, wherein the organosilicone material comprises the units (I) and (II) according to a unit (I)/unit (II) molar ratio ranging from 30/70 to 50/50.

15. The composition according to claim 14, wherein the organosilicone material comprises the units (I) and (II) according to a unit (I)/unit (II) molar ratio ranging from 35/65 to 45/55.

16. The composition according to claim 5, wherein the particles composed of the organosilicone material are formed by a process comprising:
(a) introducing into an aqueous medium, in the presence of at least one hydrolysis catalyst and optionally of at least-one surfactant, a compound (III) of formula SiX4 and a compound (IV) of formula RSiY₃, wherein
X and Y are chosen from, independently of one another, a C₁-C₄ alkoxy group, an alkoxyethoxy group (C₁-C₄ alkoxy), a C₂-C₄ acyloxy group, an N,N-dialkylamino group (C₁-C₄ alkyl ), a hydroxyl group, a halogen atom and a hydrogen atom, and
R is an organic group comprising a carbon atom connected directly to the silicon atom; and
(b) bringing the mixture resulting from (a) into contact with an aqueous solution including at least one polymerization catalyst and optionally at least one surfactant, at a temperature ranging from 30 to 85° C., for at least two hours.

17. The composition according to claim 16, wherein, in (a), the molar ratio of the compound (III) to the compound (IV) ranges from 30/70 to 50/50.

18. The composition according to claim 17, wherein in (a), the molar ratio of the compound (III) to the compound (IV) is 40/60.

19. The composition according to claim 16, wherein, in (a), the ratio by weight of water to the total of the compounds (III) and (IV) ranges from 10/90 to 70/30.

20. The composition according to claim 16, wherein the organic group is chosen from a reactive organic group and an unreactive organic group.

21. The composition according to claim 20, wherein the organic group is an unreactive organic group.

22. The composition according to claim 21, wherein the unreactive organic group is chosen from a C₁-C₄ alkyl group and a phenyl group.

23. The composition according to claim 22, wherein the unreactive organic group is a methyl group.

24. The composition according to claim 20, wherein the reactive organic group is chosen from an epoxy group, a (meth)acryloyloxy group, an alkenyl group, a mercaptoalkyl, a aminoalkyl group, a haloalkyl group, a glyceroxy group, a ureido group and a cyano group.

25. The composition according to claim 24, wherein the reactive organic group is chosen from an epoxy group, a (meth)acryloyloxy group, an alkenyl group, a mercaptoalkyl and an aminoalkyl group.

26. The composition according to claim 16, wherein R is a methyl group.

27. The composition according to claim 16, wherein the hydrolysis and polymerization catalysts are chosen independently from sodium hydroxide, potassium hydroxide, sodium carbonate, sodium hydrogencarbonate, ammonia, trimethylamine, triethylamine, tetramethylammonium hydroxide, citric acid, acetic acid, methanesulphonic acid, p-toluenesulphonic acid, dodecylbenzenesulphonic acid, dodecylsulphonic acid, hydrochloric acid, sulphuric acid and phosphoric acid.

28. The composition according to claim 1, wherein the concave particles are formed, in longitudinal cross section, of a small internal arc, of a large external arc and of segments which connect the ends of the respective arcs;
the width between the two ends of the small internal arc ranging from 0.01 to 8 μm on average;
the width between the two ends of the large external arc ranging from 0.05 to 10 μm on average; and
the height of the large external arc ranging from 0.015 to 8 μm on average.

29. The composition according to claim 28, wherein the width between the two ends of the small internal arc ranges from 0.02 to 6 μm on average.

30. The composition according to claim 28, wherein the width between the two ends of the large external arc ranges from 0.06 to 8 μm on average.

31. The composition according to claim 28, wherein the height of the large external arc ranges from 0.03 to 6 μm on average

32. The composition according to claim 1, wherein the concave particles are present in an amount ranging from 0.01% to 50% by weight relative to the total weight of the composition.

33. The composition according to claim 32, wherein the concave particles are present in an amount ranging from 1% to 15% by weight relative to the total weight of the composition.

34. The composition according to claim 1, wherein the platelet-shaped particles are particles having a greatest dimension and a thickness such that the ratio of the greatest dimension to the thickness is greater than or equal to 5.

35. The composition according to claim 1, wherein the platelet-shaped particles are chosen from platelets of inorganic materials.

36. The composition according to claim 1, wherein the platelet-shaped particles are chosen from platelets formed of mica, sericite, glass, silica, aluminium oxide and barium sulphate.

37. The composition according to claim 1, wherein the platelet-shaped particles are covered with a layer of metal or of metal oxide.

38. The composition according to claim 37, wherein the metal is chosen from silver, aluminium, chromium, nickel, molybdenum, gold, copper, tin, and magnesium and the mixtures thereof.

39. The composition according to claim 37, wherein the metal oxide is chosen from titanium dioxide, iron oxides, zinc oxides, and chromium oxide and the mixtures thereof.

40. The composition according to claim 39, wherein the metal oxide is titanium dioxide.

41. The composition according to claim 1, wherein the platelet-shaped particles are flat fibers.

42. The composition according to claim 41, wherein the flat fibers have a length L and a diameter D such that the ratio UD ranges from 5 to 2500.

43. The composition according to claim 42, wherein the ratio UD ranges from 5 to 150.

44. The composition according to claim 41, wherein the fibers have a transverse cross section included within a circle with a diameter ranging from 2 nm to 500 μm.

45. The composition according to claim 44, wherein the fibers have a transverse cross section included within a circle with a diameter ranging from 1 μm to 70 μm.

46. The composition according to claim 41, wherein the flat fibers have a length L ranging from 1 μm to 10 mm.

47. The composition according to claim 46, wherein the flat fibers have a length L ranging from 0.3 mm to 3.5 mm.

48. The composition according to claim 41, wherein the flat fibers have a transverse cross section exhibiting a greatest length L1 and a smallest length L2 such that L1/L2 is greater than 4.

49. The composition according to claim 48, wherein L1/L2 is greater than 7.

50. The composition according to claim 48, wherein L1/L2 ranges from 4 to 15.

51. The composition according to claim 48, wherein L1/L2 ranges from 7 to 10.

52. The composition according to claim 41, wherein the flat fibers have a transverse cross section of rectangular, ovoid or ellipsoidal shape.

53. The composition according to claim 41, wherein the flat fibers are in ribbon or tagliatelle form.

54. The composition according to claim 41, wherein the flat fibers have a count ranging from 0.15 to 30 deniers.

55. The composition according to claim 54, wherein the flat fibers have a count ranging from 0.18 to 18 deniers.

56. The composition according claim 41, wherein the flat fibers are fibers formed of polymers.

57. The composition according claim 56, wherein the flat fibers are chosen from fibers formed of rayon, polyarnide, viscose, acetate, poly(p-phenylene terephthalamide), acrylic polymer, polyolefin, polytetrafluoroethylene, poly(vinyl chloride), poly(vinylidene chloride), poly(vinylidene fluoride), poly(vinyl alcohol), polyacrylonitrile, polyurethane, polyesters, and polycarbonate.

58. The composition according to claim 41, wherein the flat fibers are chosen from fibers with a multilayer structure comprising alternating layers of polymers chosen from polyesters, acrylic polymers and polyamides.

59. The composition according to claim 1, wherein the platelet-shaped particles are present in an amount ranging from 1% to 99% by weight relative to the total weight of the composition.

60. The composition according to claim 59, wherein the platelet-shaped particles are present in an amount ranging from 10% to 80% by weight relative to the total weight of the composition.

61. The composition according to claim 1, wherein the platelet-shaped particles and the concave particles are present in an amount such that the ratio of the weight of the platelet-shaped particles to the weight of the concave particles ranges from 1 to 100.

62. The composition according to claim 61, wherein the platelet-shaped particles and the concave particles are present in an amount such that the ratio of the weight of the platelet-shaped particles to the weight of the concave particles ranges from 5 to 50.

63. The composition according to claim 1, wherein the concave particles are present in an amount ranging from 1% to 50% by weight relative to the total weight of the concave particles and the platelet-shaped particles.

64. The composition according to claim 63, wherein the concave particles are present in an amount ranging from 5% to 40% by weight relative to the total weight of the concave particles and the platelet-shaped particles.

65. The composition according to claim 1, further comprising an additional pulverulent coloring material different from the platelet-shaped particles and chosen from pigments and glitter.

66. The composition according to claim 65, wherein the pigments are chosen from titanium dioxide, zirconium oxide, cerium oxide, zinc oxides, iron oxides, chromium oxide, manganese violet, ultramarine blue, chromium hydrate, ferric blue, aluminium powder, copper powder, carbon black, pigments of D & C type, and lakes.

67. The composition according to claim 65, wherein the pigments are present in an amount ranging from 0.1% to 15% by weight relative to the total weight of the composition.

68. The composition according to claim 65, wherein the pigments are present in an amount ranging from 1% to 10% by weight relative to the total weight of the composition.

69. The composition according to claim 1, further comprising an additional filler different from the platelet-shaped particles.

70. The composition according to claim 69, wherein the additional filler is chosen from talc, mica, silica, kaolin, powders formed of polyarnide, powders formed of poly-β-alanine, powders formed of polyethylene, powders formed of polyurethane, powders formed of tetrafluoroethylene polymers, lauryllysine, starch, boron nitride, polymeric hollow microspheres, silicone resin microbeads, particles formed of polyorganosiloxane elastomers, precipitated calcium carbonate, magnesium carbonate, basic magnesium carbonate, hydroxyapatite, hollow silica microspheres, glass or ceramic microcapsules, and metal soaps derived from organic carboxylic acids having from 8 to 22 carbon atoms.

71. The composition according to claim 69, wherein the additional fillers are present in an amount ranging from 0.1% to 90% by weight relative to the total weight of the composition.

72. The composition according to claim 71, wherein the additional fillers are present in an amount ranging from 5% to 70% by weight relative to the total weight of the composition.

73. The composition according to claim 1, further comprising a liquid fatty phase.

74. The composition according to claim 73, wherein the liquid fatty phase comprises an oil chosen from mink oil, turtle oil, soybean oil, grape seed oil, sesame oil, maize oil, rapeseed oil, sunflower oil, cottonseed oil, avocado oil, olive oil, castor oil, jojoba oil, groundnut oil, liquid paraffins, squalane, liquid petrolatum, polydecene, isopropyl myristate, isopropyl palmitate, butyl stearate, isodecyl stearate, hexyl laurate, isononyl isononanoate, 2-ethylhexyl palmitate, 2-hexyldecyl laurate, 2-octyidecyl palmitate, 2-octyldodecyl myristate, 2-octyldodecyl lactate, di(2-ethylhexyl) succinate, diisostearyl malate, glyceryl triisostearate, diglyceryl triisostearate, silicone oils, fluorinated silicones, perfluorinated oils, oleic acid, linoleic acid, linolenic acid, cetanol and oleyl alcohol.

75. The composition according to claim 73, wherein the liquid fatty phase is present in an amount ranging from 0.1% to 13% by weight relative to the total weight of the composition.

76. The composition according to claim 75, wherein the liquid fatty phase is present in an amount ranging from 0.1% to 8% by weight relative to the total weight of the. composition

77. The composition according to claim 1, further comprising a cosmetic ingredient chosen from waxes, preservatives, cosmetic active principles, moisturizing agents, UV screening agents, thickeners, water, surfactants and fragrances.

78. The composition according to claim 1, wherein the composition is provided in the form of a compact powder.

79. The composition according to claim 1, wherein the composition is in the form of a blusher, an eyeshadow, a face powder, a foundation, a concealer, a product for making up the body, a product for caring for the face, a product for caring for the body or an antisun product.

80. A cosmetic process for making up or for non-therapeutic treatment of keratinous substances comprising applying a composition to the keratinous substances, wherein the composition comprises a pulverulent phase comprising concave particles and particles having the shape of platelets.
